(19) United States
(12) Patent Application Publication
(10) Pub. No.: US 2025/0041577 A1
(43) Pub. Date: Feb. 6, 2025

(54) SHORT-PULSE SONODYNAMIC TREATMENT APPARATUS

(71) Applicant: Insightec, Ltd., Tirat Carmel (IL)

(72) Inventors: Yeruham SHAPIRA, Rehovot (IL); Rafi DE PICIOTTO, Carmei Yosef (IL); Yoav LEVY, Hinanit (IL); Stuart L. MARCUS, Berkeley, CA (US)

(21) Appl. No.: 18/718,304

(22) PCT Filed: Dec. 9, 2022
(86) PCT No.: PCT/IB2022/000750

§ 371 Date: Jun. 10, 2024

Related U.S. Application Data
(60) Provisional application No. 63/288,428, filed on Dec. 10, 2021.
(60) Provisional application No. 63/288,436, filed on Dec. 10, 2021.

(51) Int. Cl. A61M 37/00; A61K 41/00

(52) U.S. Cl. CPC A61M 37/0092; A61K 41/0047; A61M 2037/0007

(57) ABSTRACT

Sonodynamic therapy systems and methods use short, high-pressure ultrasound pulses that produce small (and therefore clinically safe) increases (up to 1-2° C.) in tissue temperature but nonetheless activate sonodynamic agents, allowing isolated single-cav-itation events to produce therapeutically beneficial effects (e.g., cytotoxicity) on diseased tissue. An escalating sonication strategy may be employed whereby the focal pressure is gradually increased until maximum clinically tolerable parameters such as the degree of heating of intervening tissue (e.g., the skull when treating brain cancer) or undesired induced cavitation effects are reached.

### RELATED APPLICATION

This application claims the benefit of and priority to U.S. Provisional Patent Application Nos. 63/288,428 and 63/288,436 (both filed on Dec. 10, 2021), the entire disclosures of which are hereby incorporated by reference.

### FIELD

The present invention relates, generally, to ultrasound procedures, and more particularly to systems and methods for using focused ultrasound in combination with photosensitization.

### BACKGROUND

A common strategy for the treatment of certain cancers is administration of photosensitive agents with affinity for tumor cells, followed by exposure of the tumor location to stimulating light. Research suggests that the use of such agents in combination with specific sequences of focused ultrasound (FUS) pulses may also yield therapeutic benefits against the cancerous tissue, although the underlying mechanism for the observed effects remains uncertain. Possibilities include a minute tissue temperature rise of less than 2° C., exposure to low-magnitude pressure fields, and/or cavitation effects that may trigger photon emission. The mechanism of action has not been conclusively determined, but is believed to be due to thermal and mechanical effects, and/or singlet oxygen produced by cavitation. Ultrasound is capable of penetrating tissue to a far greater distance than light, making more of the body accessible to non-invasive treatment. This approach has been termed sonodynamic therapy (“SDT”) and a sensitizer activated by FUS is often called a sonosensitizer. Agents that have been employed or investigated include texaphyrins, tetrasulphamoylphthalo-cyanine and naphthalocyanine derivatives.

Efforts to investigate the clinical effectiveness of sonodynamic therapy have generally used low-pressure fields (about 100-500 kPa) and/or procedures giving rise to small increases (1-2° C.) in tissue temperature, and have been shown to yield some therapeutic effects in preclinical work.

### BRIEF SUMMARY

Embodiments described herein use a train of very short, high-pressure ultrasound pulses that produce small (and therefore clinically safe) increases (1-2° C.) in tissue temperature but nonetheless activate sonodynamic agents, allowing isolated single-cavitation events to produce therapeutically beneficial effects (e.g., cytotoxicity) on diseased tissue. A sonication strategy may be employed whereby the maximum clinically tolerable focal pressure is employed, e.g., with respect to the resulting degree of heating of intervening tissue (e.g., the skull when treating brain cancer) or undesired induced cavitation effects.

For example, a sonodynamic agent such as 5-aminolevulinic acid or fluorescein may be introduced (parenterally or orally) to the diseased tissue, and the tissue may be exposed to ultrasound energy (“sonicated”) using a focused ultrasound device at a pulse duration of from about 0.1 millisec to about 100 millisec, at a pulse repetition frequency from about 0.1 Hz to about 50 Hz, at an intensity at the ultrasound beam focus from about 5 W/cm² to about 10,000 W/cm² (or as high as can be tolerated without significant clinical effect), and a total duration of the pulse sequence of 10 sec to 1200 sec. As used herein, the term “significant clinical effect” means having an adverse (sometimes, absent of a desired) effect on a tissue that is considered significant by a clinician, e.g., the onset of damage to the tissue or other clinically adverse effects, whether temporary or permanent.

### BRIEF DESCRIPTION OF THE DRAWINGS

FIG. 1 schematically illustrates a microbubble-mediated focused ultrasound system in accordance with various embodiments.

FIG. 2 shows plasma and blood pharmacokinetics of 5-aminolevulinic acid (“5-ALA”) and protoporphyrin-IX (“PPIX”) following 10 mg/kg of IV Sonala-001. Concentration (nM) vs. time (hours) is noted. Elevated PPIX levels were exhibited within 15 min of dosing and a peak at approximately 6 hours. Both 5-ALA and PPIX returned to endogenous levels 24 hours post 5-ALA dose administration.

FIG. 3 shows measures of reactive oxygen species, as a measure of free glutathione to glutathione disulfide (“GSH/GSSG”), free cysteine to cysteine disulfide (“Cys/CySS”), or Total Thiol in an untreated patient (“Control”) or after sonodynamic therapy (“SDT”) in Patient 1.

FIG. 4 shows tumor pharmacodynamics for three recurrent glioblastoma patients treated with 5-ALA sonodynamic therapy. In 5-ALA SDT-treated tumor regions of the three patients, a significant increase in (A) enhanced tumor cell death (as measured by C1-Cas3 (%) in controls (“CON”) and treated patients (“SDT”)) was accompanied by targeted reactive oxygen species formation, as measured by (B) 4-HNE levels in control (“CON”) and treated patients (“SDT”).

FIG. 5 shows tumor pharmacodynamics for three recurrent glioblastoma patients treated with 5-ALA sonodynamic therapy as measured by (A) 2GSH/GSSG ratio, (B) 2Cys/CySS ratio, and (C) Total Thiol levels in controls (“CON”) and treated patients (“SDT”).

### DETAILED DESCRIPTION

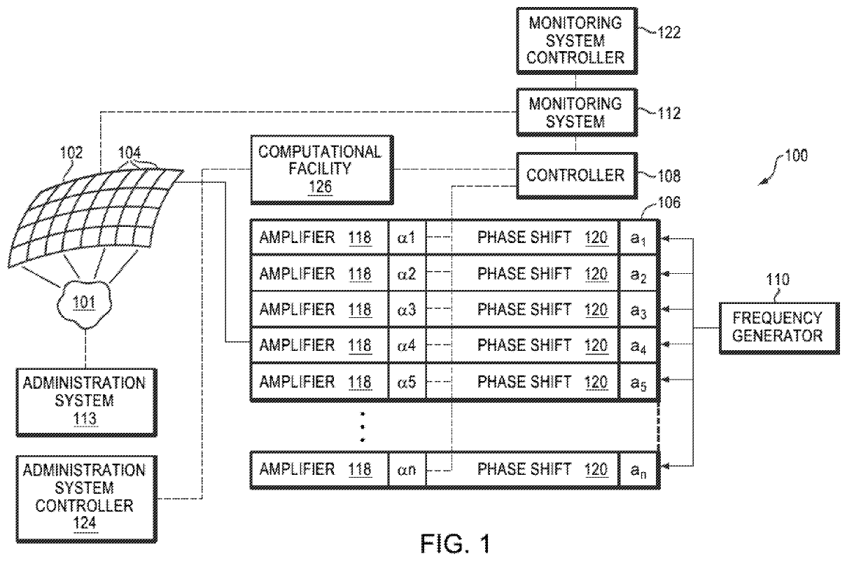

FIG. 1 illustrates an exemplary ultrasound system 100 for generating and delivering a focused acoustic energy beam to a target region 101 within a patient's body. The illustrated system 100 includes a phased array 102 of transducer elements 104, a beamformer 106 driving the phased array 102, a controller 108 in communication with the beamformer 106, and a frequency generator 110 providing an input electronic signal to the beamformer 106. In various embodiments, the system further includes a monitoring system 112 for detecting information about the patient and an administration system 113 for introducing a sonosensitizer into the patient's body as further described below. The monitoring system 112 and administration system 113 may be operated using the same controller 108 that facilitates the transducer operation; alternatively, they may be separately controlled by two separate controllers 122, 124, respectively. The controllers 108, 122, 124 may intercommunicate.

The array 102 may have a curved (e.g., spherical or parabolic) or other contoured shape suitable for placement on the surface of the patient's body, or may include one or more planar or otherwise shaped sections. Its dimensions may vary between millimeters and tens of centimeters. The transducer elements 104 of the array 102 may be piezoelectric ceramic elements, and may be mounted in silicone rubber or any other material suitable for damping the mechanical coupling between the elements 104. Piezo-composite materials, or generally any materials or combination of materials (as in MEMS devices) capable of converting electrical energy to acoustic energy, may also be used. To assure maximum power transfer to the transducer elements 104, the elements 104 may be configured so that the electrical resonance matches the input connector impedance (e.g., 50 Ω).

The transducer array 102 is coupled to the beamformer 106, which drives the individual transducer elements 104 so that they collectively produce a focused ultrasonic beam or field. For n transducer elements, the beamformer 106 may contain n (or fewer) driver circuits, each including or consisting of an amplifier 118 and a phase delay circuit 120; each drive circuit drives one or more of the transducer elements 104. The beamformer 106 receives a radio frequency (RF) input signal, typically in the range from 0.1 MHz to 10 MHz, from the frequency generator 110, which may, for example, be a Model DS345 generator available from Stanford Research Systems. The input signal may be split into n channels for the n amplifiers 118 and delay circuits 120 of the beamformer 106. In some embodiments, the frequency generator 110 is integrated with the beamformer 106. The radio frequency generator 110 and the beamformer 106 are configured to drive the individual or sets of transducer elements 104 of the transducer array 102 at the same frequency, but at different phases and/or different amplitudes.

The amplification or attenuation factors α₁-αₙ and the phase shifts a₁-aₙ imposed by the beamformer 106 serve to transmit and focus ultrasonic energy through the intervening tissue located between the transducer elements 104 and the target region onto the target region 101, and account for wave distortions induced in the intervening tissue. The amplification factors and phase shifts are computed using the controller 108, which may provide the computational functions through software, hardware, firmware, hardwiring, or any combination thereof. In various embodiments, the controller 108 utilizes a general-purpose or special-purpose digital data processor programmed with software in a conventional manner, and without undue experimentation, in order to determine the phase shifts and amplification factors necessary to obtain a desired focus or any other desired spatial field patterns. The determined phase shifts and amplification factors together with other acoustic parameter(s) may then be included in a treatment plan for treating the target region 101 as further described below.

In certain embodiments, the monitoring system 112 includes an imager, such as a magnetic resonance imaging (MRI) device, a computer tomography (CT) device, a positron emission tomography (PET) device, a single-photon emission computed tomography (SPECT) device, or an ultrasonography device, for determining characteristics (e.g., the type, property, structure, size, location, shape, temperature, etc.) of the tissue at the target region 101 and/or the non-target region.

To determine a suitable acoustic power profile for treatment, in various embodiments, a tissue model is used to characterize the material properties of the target and/or non-target tissue, such as their heat sensitivities and/or tolerable thermal energies. The treatment plan then specifies one or more therapeutic objectives including a disruption level at the target region 101 and/or a maximum tolerable temperature, an acoustic energy and/or an acoustic response (e.g., a cavitation dose) at the non-target region. A physical model that simulates ultrasound field aberrations resulting from, for example, beams traversing inhomogeneous intervening tissue located between the transducer 102 and the target tissue 101, transducer geometry and/or acoustic field design (e.g., for refocusing purposes) can be used to inversely compute the required acoustic power and/or other parameter values (e.g., amplitudes, frequencies, phases, directions and/or activation time associated with the transducer elements, or time intervals between consecutive series of sonications) associated with the transducer elements 104 based on the location and disruption temperature of the target region 101 and/or the maximum tolerable temperature at the non-target region. Further details about establishing the tissue model and physical model are provided, for example, in International Application No. PCT/IB2017/001689 (filed on Dec. 13, 2017), International Patent Publication No. WO 2018/130867, and U.S. Patent Publication No. 2015/0359603, the entire disclosures of which are hereby incorporated herein by reference. In addition, approaches to computationally generating a treatment plan using the tissue model and physical model are provided in International Application No. PCT/IB2018/000834 (filed on Jun. 29, 2018), the entire disclosure of which is hereby incorporated herein by reference.

Additionally or alternatively, the monitoring system 112 may include one or more biosensors for measuring one or more physiological parameters (e.g., blood pressure, blood circulation rate, blood perfusion rate, and/or heart rate) of the patient. The monitoring system 112 may be controlled by the ultrasound controller 108, or alternatively, a separate controller 122. For example, the monitoring system 112 may be a standard heart rate measurement system with an internal controller 122 that is operated manually. The controller 122 may be in communication with the ultrasound controller 108 and/or a computational facility 126 to provide the measured physiological parameter(s) thereto prior to and/or during the ultrasound procedure. The computational facility 126 facilitates treatment planning and adjustment and may take the measured physiological parameter(s) into account when determining and/or modifying the treatment plan as further described below.

In a typical procedure, a sonodynamic agent such as 5-ALA or fluorescein is introduced, parenterally (e.g., via the administration system 113) or orally, so as to reach the diseased tissue, and the target tissue is sonicated using the system 100. Representative sonication parameters include pulse duration of from about 0.1 millisec to about 100 millisec, a pulse repetition frequency from about 0.1 Hz to about 50 Hz, an intensity at the ultrasound beam focus from about 5 W/cm² to about 10,000 W/cm², and a total duration of the pulse sequence of 10 sec to 1200 sec. Because the treatment objective is to ensure a cytotoxic effect at the target region, the intensity at the ultrasound beam focus may be as high as can be tolerated without significant clinical effect as indicated, for example, by the physical model as described above. The physiological response may be monitored by the monitoring system 112 to ensure that safety constraints are not exceeded.

The appropriate amount of sonosentizer to be administered can be determined by standard methods. In general, the effective amount will be an amount sufficient to substantially stain the malignant tissue to be treated, without substantially staining normal tissue, or inducing an unacceptable level of toxicity. Without being bound by any particular theory, it is believed that sonicating tissue causes spontaneous generation of microbubbles and their cavitation, the collapse of which causes generation of photons having wavelengths between about 300 nm and 700 nm within the tissue, and that either these photons or the application of the original pressure wave itself activates the sonosensitizer, leading to tissue destruction.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both limits; ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, etc. As a non-limiting example, each range discussed herein can be readily broken down into a lower third, middle third and upper third, and so forth. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is to be appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

### Malignant Tissue

Malignant tissue is typically tumorous or cancerous, but in general may be any type of tissue that is capable of taking up a sonosensitizer rapidly compared to normal tissues (e.g., absorbing 5-ALA and accumulating protoporphyrin-IX), for example, a benign tumor or other unwanted growth. Focused ultrasound is capable of passing through intervening tissue, which enables treatment of malignant tissue situated at otherwise inaccessible positions. For this reason, the systems and methods described herein are particularly useful for treating types of intracranial tumors, such as glioblastoma multiforme (including low-grade and high-grade glioblastomas), optical pathway gliomas, diffuse intrinsic pontine gliomas, astrocytoma, ependymoma, medulloblasoma, oligodendroglioma, hemangioblastoma, rhabdoid tumors, brain metastases from other cancers (including, for example without limitation, breast adenocarcinoma, small cell lung carcinoma, non-small cell lung carcinoma, squamous cell lung carcinoma, metastatic malignant melanoma, and prostate carcinoma), meningioma, primary pituitary gland malignancies, malignant nerve sheath tumors, and neurofibromas. Other malignant tissues include, without limitation, neoplasms, carcinomas, sarcomas, leukemias, lymphomas, and the like. Leukemias and lymphomas include, for example, cutaneous T-cell lymphoma (CTCL), noncutaneous peripheral T-cell lymphoma, lymphomas associated with human T-cell lymphotropic virus (HTLV), for example, adult T-cell leukemia/lymphoma (ATLL), acute lymphocytic leukemia, acute nonlymphocytic leukemias, chronic lymphocytic leukemia, chronic myelogenous leukemia, Hodgkin's Disease, non-Hodgkin's lymphomas, and multiple myeloma. Other tumors include, without limitation, childhood solid tumors such as brain tumors, neuroblastoma, retinoblastoma, Wilms' Tumor, bone tumors, and soft-tissue sarcomas; common solid tumors of adults such as head and neck cancers (e.g., infiltrating or metastatic squamous cell carcinoma, salivary gland tumors, nasopharyngeal carcinomas, oral, laryngeal, and esophageal tumors); genitourinary cancers (e.g., urethral, ureteral, renal cell, bladder carcinoma and bladder carcinoma in situ, locally advanced or metastatic carcinoma of the prostate, bladder, renal, uterine, ovarian, testicular, cancers, uterine, cervical, and uterine carcinoma), rectal, and colon cancer; lung cancer (including mesothelioma, small cell lung carcinoma, non-small cell lung carcinoma, squamous cell lung carcinoma); breast cancer; gastric, esophageal, and colon carcinoma, cholangiocarcinoma, hepatic carcinoma, and pancreatic adenocarcinoma; melanoma, infiltrating basal cell carcinomas, and other skin cancers; stomach cancer, brain cancer, liver cancer and thyroid cancer.

In particular uses and implementations, the malignant tissue is glioblastoma multiforme, optical pathway glioma, diffuse intrinsic pontine glioma, astrocytoma, ependymoma, medulloblasoma, oligodendroglioma, hemangioblastoma, rhabdoid tumor, brain metastases from another cancers (such as breast adenocarcinoma, small cell lung carcinoma, non-small cell lung carcinoma, squamous cell lung carcinoma, metastatic malignant melanoma, or prostate carcinoma), meningioma, primary pituitary gland malignancy, malignant nerve sheath tumor, neurofibroma, cutaneous T-cell lymphoma (CTCL), noncutaneous peripheral T-cell lymphoma, lymphomas associated with human T-cell lymphotropic virus (HTLV), adult T-cell leukemia/lymphoma (ATLL), acute lymphocytic leukemia, acute nonlymphocytic leukemias, chronic lymphocytic leukemia, chronic myelogenous leukemia, Hodgkin's Disease, non-Hodgkin's lymphomas, multiple myeloma, neuroblastoma, retinoblastoma, Wilms' Tumor, bone tumors, soft-tissue sarcomas, infiltrating or metastatic squamous cell carcinoma, salivary gland tumors, nasopharyngeal carcinomas, oral, laryngeal, esophageal tumors, urethral cancer, ureteral cancer, renal cell cancer, bladder carcinoma, bladder carcinoma in situ, metastatic carcinoma of the prostate, bladder, renal, uterine, ovarian, testicular, cancers, uterine, cervical, and uterine carcinoma, rectal or colon cancer, lung cancer, mesothelioma, small cell lung carcinoma, non-small cell lung carcinoma, squamous cell lung carcinoma, breast cancer; gastric cancer, esophageal cancer, and colon carcinoma, cholangiocarcinoma, hepatic carcinoma, pancreatic adenocarcinoma, melanoma, infiltrating basal cell carcinomas, other skin cancers, liver cancer or thyroid cancer. In some embodiments, the malignant tissue is a glioblastoma multiforme, astrocytoma, ependymoma, medulloblasoma, oligodendroglioma, hemangioblastoma, or a rhabdoid tumor. In some embodiments, the malignant tissue is glioblastoma multiforme.

### 5-Aminolevulinic Acid

5-ALA can be provided in any pharmaceutically acceptable formulation, and may be provided as the free acid, or a pharmaceutically acceptable salt thereof. A formulation, GLIOLAN, is commercially available. Salts, esters, amides, prodrugs and other derivatives of the active agents can be prepared using standard procedures known to those skilled in the art of synthetic organic chemistry and described, for example, by March (1992), Advanced Organic Chemistry; Reactions, Mechanisms and Structure, 4th Ed. N.Y. Wiley-Interscience. Pharmaceutically acceptable salts are salts that retain the biological effectiveness and properties of the parent compound and which are not biologically or otherwise undesirable. 5-ALA can form acid and/or base salts by virtue of the presence of amino and/or carboxyl groups. Many such salts are known in the art, for example, as described in published PCT Appl. No. WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine.

In some embodiments, the 5-ALA is sterilized by gamma irradiation (see, e.g., U.S. Pat. No. 6,335,465, incorporated herein by reference in full). The sonosensitizer (e.g., 5-ALA or fluorescein) formulation can be administered orally, intravenously (e.g., via the administration system 113), intrathecally, or intratumorally. In some embodiments, the sonosensitizer (e.g., 5-ALA or fluorescein) is administered at a dosage of at least about 1, 2, 3, 4, 5, 6, 7, 8, 9, 10, 12, 15, 20, 25, 30, 35, 40, 45, 50, 55, 60, 65, 70, 75, 80, 85, 90, 95, 96, 97, 98, 99, 100, 120, 125, 150, 175, 200, 300, 400, 500, 600, 750, or at least about 1000 mg/kg. In some embodiments, the sonosensitizer (e.g., 5-ALA or fluorescein) is administered at a dosage of no more than about 1000, 900, 800, 700, 600, 500, 400, 300, 250, 200, 180, 175, 160, 150, 140, 130, 120, 110, 100, 90, 80, 70, 60, 50, 45, 40, 35, 30, 25, or 20 mg/kg. For example, the sonosensitizer (e.g., 5-ALA or fluorescein) may be administered at a dosage of about 0.5 to about 250 mg/kg, at a dosage of 1 to 150 mg/kg, at a dosage of 5 to 90 mg/kg, or at a dosage of 10 to 40 mg/kg.

The effective amount of sonosensitizer required can be determined by standard methods known to those of skill in the art. For example, as described in Example 1 herein, one can treat a human subject with an appropriate amount of 5-ALA and different FUS conditions to treat glioblastoma. As shown in the Examples, human subjects can be treated with 10 mg/kg of 5-ALA to be converted to protoporphyrin-IX. In some embodiments, the effective amount of 5-ALA is between 1 mg/kg and 1,000 mg/kg. In some embodiments, the effective amount of 5-ALA is between 5 mg/kg and 750 mg/kg. In some embodiments, the effective amount of 5-ALA is between 10 mg/kg and 750 mg/kg. In some embodiments, the effective amount of 5-ALA is between 20 mg/kg and 500 mg/kg. In some embodiments, the effective amount of 5-ALA is between 40 mg/kg and 500 mg/kg. In some embodiments, the effective amount of 5-ALA is between 5 mg/kg and 40 mg/kg. In some embodiments, the effective amount of 5-ALA is between 5 mg/kg and 20 mg/kg. In some embodiments, the effective amount of 5-ALA is about 5 mg/kg, about 10 mg/kg, about 15 mg/kg, or about 20 mg/kg. In some embodiments, the effective amount of 5-ALA is about 10 mg/kg.

An incubation period may be included between administering a sonosensitizer and sonicating the malignant tissue, to allow sufficient time for the sonosensitizer to be taken up by the malignant tissue (and, in the case of 5-ALA, converted to protoporphyrin-IX). In some embodiments, the incubation period is at least about 30 minutes, at least about one hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 18 hours, at least about 20 hours, or at least about 24 hours. In some embodiments, the incubation period is 72 hours, less than about 72 hours, less than about 60 hours, less than about 48 hours, less than about 36 hours, less than about 24 hours, less than about 22 hours, less than about 20 hours, less than about 18 hours, less than about 16 hours, less than about 15 hours, less than about 14 hours, less than about 13 hours, less than about 12 hours, less than about 11 hours, less than about 10 hours, less than about 9 hours, less than about 8 hours, less than about 7 hours, less than about 6 hours, less than about 5 hours, less than about 4 hours, or less than about 3 hours. In some embodiments, the incubation period is between 1 and 72 hours. In some embodiments, the incubation period is between 2 and 48 hours. In some embodiments, the incubation period is between 3 and 36 hours. In some embodiments, the incubation period is between 4 and 24 hours. In some embodiments, the incubation period is between 4 and 18 hours. In some embodiments, the incubation period is between 4 and 24 hours. In some embodiments, the incubation period is between 4 and 18 hours. In some embodiments, the incubation period is about 6 hours.

### Potentiating Agents

In some embodiments, the method further comprises administering a potentiating agent that enhances the therapeutic effect of the sonosensitizer, for example, in the case of 5-ALA, by promoting or increasing the uptake or accumulation of protoporphyrin-IX and/or 5-ALA, decreasing the rate at which protoporphyrin-IX and/or 5-ALA is metabolized, and the like. The potentiating agent can thus reduce the amount of sonosensitizer required in order to obtain a given effect or can increase the effect obtained from a given amount of sonosensitizer, or any combination of desired effect and amount in between. Suitable potentiating agents include, for example without limitation, methotrexate, doxycycline, minocycline, Vitamin D3 and derivatives thereof. See, e.g., D.-F. Yang et al., J Formos Med Assoc (2014) 113 (2): 88-93; M.-J. Lee et al., PLoS ONE (2017) 12 (5): e0178493; and E. V. Maytin et al., Isr J Chem (2012) 52 (8-9): 767-75. In some embodiments, the potentiating agent is selected from the group consisting of methotrexate, doxycycline, minocycline, Vitamin D₃ and derivatives thereof. In some embodiments, the potentiating agent is methotrexate. In some embodiments, the potentiating agent is doxycycline. In some embodiments, the potentiating agent is minocycline. In some embodiments, the potentiating agent is Vitamin D₃. In some embodiments, a combination of two or more potentiating agents is used. In some embodiments, a combination of two or more of methotrexate, doxycycline, minocycline, and Vitamin D₃ is used.

The potentiating agent can be administered at the same time as the sonosensitizer, or at any other time prior to sonication. The optimal time for administering a potentiating agent can vary with the selection of potentiating agent or combination of agents. In some embodiments, the potentiating agent is administered at the same time as the sonosensitizer. In some embodiments, the potentiating agent is administered in the same formulation as the sonosensitizer. In some embodiments, the potentiating agent is administered at a different time. In some embodiments, the potentiating agent is administered prior to sonosensitizer administration. In some embodiments, the potentiating agent is administered at least about 30 minutes, at least about one hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 18 hours, at least about 20 hours, at least about 24 hours prior to the first sonication. In some embodiments, the potentiating agent is administered at about 24 hours, about 22 hours, about 20 hours, about 18 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, or about 3 hours prior to the first sonication. In some embodiments, the potentiating agent administration period is between 1 and 72 hours prior to sonication. In some embodiments, the potentiating agent administration period is between 2 hours and 5 days prior to sonication. In some embodiments, the potentiating agent administration period is between 18 hours and 4 days prior to sonication. In some embodiments, the potentiating agent administration period is between 24 hours and 4 days prior to sonication. In some embodiments, the potentiating agent administration period is between 4 and 18 hours prior to sonication. In some embodiments, the potentiating agent is administered at about 6 hours prior to the first sonication.

The amount of potentiating agent administered can be determined by those of skill in the art and will in general depend on the potentiating agent or agents selected and the degree of potentiating effect to be obtained. Suitable methods include, for example without limitation, cell culture assays and/or in vivo experiments with model animals or explanted tissues to determine the degree of cell killing using varying amounts of 5-ALA and/or potentiating agents, with either sonication or photodynamic treatment. See, e.g., D.-F. Yang et al., J Formos Med Assoc (2014) 113 (2): 88-93; M.-J. Lee et al., PLoS ONE (2017) 12 (5): e0178493; and E. V. Maytin et al., Isr J Chem (2012) 52 (8-9): 767-75.

The amount of potentiating agent used will be less than the amount at which unacceptable toxicity is experienced and will be large enough to decrease the amount of sonosensitizer required to obtain a potentiated effect. For example, one can determine the amount or number of malignant tissue or cells killed using a set amount of sonosensitizer as a baseline for comparison, and then determine the amount or number of malignant tissue or cells killed using the same amount of sonosensitizer in combination with different concentrations or amounts of the potentiating agent. Alternatively, one can determine the amount of sonosensitizer needed to produce the same level of killing in the presence of different concentrations or amounts of the potentiating agent. The amount or number of malignant tissue or cells killed can be determined by cell counting, measurement of tumor volume, vital dye exclusion, and other techniques commonly used in medical research. The effect obtained with the potentiating agent will be an increase in effect or a decrease in sonosensitizer dose of at least 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% from the baseline measure. In some embodiments, the effect obtained with the potentiating agent is an increase in effect of at least 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99, 100, 120, 125, 150, 175, 200, 300, 400, or 500% from the baseline measure of degree of killing. In some embodiments, the effect obtained with the potentiating agent is a decrease in the amount of sonosensitizer required to obtain the baseline killing rate of at least 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99%.

The amount of potentiating agent can be greater, equal, or less than the amount that is normally or typically prescribed for use of the potentiating agent alone. The upper limit is that amount at which unacceptable toxicity is experienced, either alone or in combination with sonosensitizer. The lower limit is the amount needed to obtain a measurable potentiation effect, and can be 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99, 100, 120, 125, 150, 175, or 200% of the typical dose. For example without limitation, methotrexate can be administered as a single oral dose of about 7.5 mg to 10 mg between 24 hours and 72 hours prior to sonication; doxycycline can be administered BID at a dose of 100 mg, beginning with a 200 mg initial loading dose, starting two to four days prior to sonication; minocycline can be administered BID at a dose of 50 to 100 mg, starting two to four days prior to sonication; and Vitamin D3 can be administered as cholecalciferol at a dose of 10,000 to 100,000 IU/day for two to four days prior to sonication.

### Microbubbles

Microbubbles (also known as microspheres) are gas-filled spheres having a diameter on the order of about 1 to 5 μm. They are sometimes used as contrast agents in medical sonography, as their echogenic properties help distinguish liquid-filled vessels from surrounding tissues. See, e.g., P. A. Dijkmans et al., Eur J Cardiology (2004) 5:245-56. The gas is often air, nitrogen, sulfur hexafluoride, or a perfluorocarbon such as, for example, octafluoropropane. The shell of the microbubble is often albumin, galactose, lipid, or a polymer. In an ultrasound acoustic field, microbubbles undergo linear oscillation at low power and non-linear oscillation at higher power, leading to rupture at high power. The frequencies at which microbubbles resonate are determined primarily by the choice of gas in the core, and the mechanical properties of the shell. Nevertheless, microbubbles can be stimulated to oscillate at a wide range of ultrasound frequencies that differ from their innate resonance frequency. Mixtures of two or more different types of microbubbles can also be used. In the practice of embodiments of the present invention, microbubbles can be used to cause cavitation (and thus target cell death) at lower acoustic power than would otherwise obtain. When administered during treatment, microbubbles allow the use of lower ultrasound intensity levels to achieve a desired therapeutic effect, as the pressure level and cavitation effects at the target are enhanced by their presence. Furthermore, microbubbles have been shown to promote sonoluminescence and promote photon-multiplier tube detection at 2-3 times the amount obtained in water under the same parameters. In some embodiments, an effective amount of microbubbles is provided to the malignant tissue.

An effective amount of microbubbles is a quantity sufficient to increase the direct cytotoxic effect of sonosensitizer and FUS on malignant tissue by at least 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99, 100, 120, 125, 150, 175, 200, 300, 400, or 500% from the baseline measure of degree of killing. Alternatively, the effective amount of microbubbles can be expressed as the quantity sufficient to decrease the sonosensitizer dose and/or FUS dose by at least 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% from the baseline measure.

Microbubbles can be prepared by methods known in the art, or can be obtained from commercial sources. Suitable microbubbles include, without limitation, enhanced contrast ultrasound microbubbles such as DEFINITY perflutren lipid microbubbles (Lantheus Medical Imaging, N. Billerica, MA), LEVOVIST lipid/galactose microspheres (Schering), OPTISON microbubbles (GE Healthcare), Sono Vue microbubbles (Bracco Diagnostics Inc.) and LUMASON microbubbles (Bracco Imaging, Monroe Township, NJ). In some embodiments, the microbubbles are enhanced contrast ultrasound microbubbles. In some embodiments, the microbubbles comprise sulfur hexafluoride or a perfluorocarbon. In some embodiments, the perfluorocarbon is octafluoropropane or perfluorohexane. In some embodiments, the microbubbles comprise air or nitrogen. In some embodiments, the microbubble shell comprises albumin.

Microbubbles can be administered together with the sonosensitizer and/or a potentiating agent, depending on the half-life of the microbubbles in the subject's system. In general, many microbubble agents have a very short half-life in human circulation, and accordingly are typically administered shortly before sonication. The quantity administered and the mode of administration is similar to the quantity and mode used by those of skill in the art when administering microbubbles for purposes of contrast-enhanced ultrasound sonography. The quantity administered will be at least 5, 10, 15, 20, 25, 30, 35, 40, 45, 50, 55, 60, 65, 70, 75, 80, 85, 90, 95, 100, 105, 110, 120, 140, 150, 160, 180, 200, 250, 300, 350, or 400% of the quantity used or recommended for use as a contrast-enhanced ultrasound sonography agent. The quantity administered will be no more than 500, 450, 400, 350, 300, 250, 200, 150, 100, 90, 80, 75, 70, 60, 50, 45, 40, 35, 30, 25, or 20% of the quantity used or recommended for use as a contrast-enhanced ultrasound sonography agent.

### Focused Ultrasound

The diseased tissue is exposed to focused ultrasound energy using a focused ultrasound device. Suitable devices include the EXABLATE system (Insightec, Dallas, TX), and the like. The EXABLATE Model 4000 Type-2 has a dedicated 1000-element transducer that can operate in a sonication mode (i.e., a focused ultrasound pressure wave delivery mode) that uses short pulses and low duty cycles to generate “pulsed sonication” at a desired power level. This pulsed sonication mode enables the device to induce stable cavitation when used in conjunction with microbubbles at much lower energy levels than ultrasound-induced cavitation. The oscillation of the microbubbles induces a well targeted, temporary, and reversible, but stable blood brain barrier disruption. The hallmark feature of the EXABLATE device is its ability to monitor thermal, and acoustic feedback in real-time to ensure a safe and effective BBB disruption. The EXABLATE device is a magnetic resonance-guided focused ultrasound (MRgFUS) device, hence, it utilizes real time MR imaging to assess and monitor the safety of the procedure.

The ultrasound frequency may be at least about 0.1 MHz, at least about 0.2 MHz, at least about 0.25 MHz, at least about 0.3 MHz, at least about 0.4 MHZ, at least about 0.45 MHZ, at least about 0.5 MHz, at least about 0.55 MHz, at least about 0.6 MHz, at least about 0.65 MHZ, at least about 0.7 MHz, at least about 0.75 MHz, at least about 0.8 MHz, at least about 0.85 MHz, at least about 0.9 MHZ, at least about 0.95 MHZ, at least about 1 MHZ, at least about 1.1 MHZ, at least about 1.5 MHz, at least about 2.0 MHz, at least about 2.1 MHZ, at least about 2.2 MHZ, at least about 2.3 MHz, at least about 2.4 MHZ, at least about 2.5 MHZ, at least about 2.75 MHz, at least about 3.0 MHz, at least about 3.5 MHz, at least about 4.0 MHz, at least about 4.5 MHz, at least about 5.0 MHz, at least about 6.0 MHz, at least about 7.0 MHz, at least about 8.0 MHz, at least about 9.0 MHz, or at least about 10.0 MHz. The ultrasound frequency is no more than about 20 MHz, no more than about 15 MHz, no more than about 10 MHz, no more than about 9.0 MHz, no more than about 8.0 MHz, no more than about 7.0 MHz, no more than about 6.0 MHz, no more than about 5.0 MHz, no more than about 4.0 MHz, no more than about 3.0 MHz, no more than about 2.8 MHz, no more than about 2.6 MHz, no more than about 2.5 MHz, no more than about 2.4 MHz, no more than about 2.3 MHz, no more than about 2.2 MHz, no more than about 2.1 MHz, or no more than about 2.0 MHz.

The focused ultrasound intensity, at the ultrasound beam focus, is from about 5 W/cm² or as low as needed to elicit a sonoluminescent effect to about 10,000 W/cm² or more, e.g., as high as can be tolerated without significant clinical effect (e.g., skull heating and indiced undesired cavitation). Representative operating power levels can range from about 5 W/cm² to about 10 W/cm², 10 W/cm² to about 20 W/cm², 20 W/cm² to about 40 W/cm², from about 50 W/cm² to about 100 W/cm², or from about 200 W/cm² to about 800 W/cm², from about 1000 W/cm² to about 5000 W/cm², from about 1000 W/cm² to about 3000 W/cm², or from about 2000 W/cm² to about 3000 W/cm². The focused ultrasound intensity at the ultrasound beam focus may be about 20 W/cm², about 50 W/cm², about 100 W/cm², about 200 W/cm², about 500 W/cm², about 1000 W/cm², about 2000 W/cm², about 2200 W/cm², about 2400 W/cm², about 2600 W/cm², about 2800 W/cm², about 3000 W/cm², about 4000 W/cm², about 5000 W/cm², about 6000 W/cm², about 7000 W/cm², about 8000 W/cm², about 9000 W/cm², or about 10,000 W/cm². For example, the intensity may range from about 5 W/cm² to about 5000 W/cm², or in some cases from about 50 W/cm² to about 5000 W/cm², or from about 100 W/cm² to about 3000 W/cm². In some embodiments, the focused ultrasound intensity is the spatial peak temporal average intensity (I_{SPTA}).

The FUS energy applied by the transducer during sonodynamic treatment is in general less than the amount of energy when using FUS to ablate tissue and may be further reduced when microbubbles are administered prior to sonication. In some embodiments, the FUS energy applied by the transducer is at least 10, 20, 30, 40, 50, 60, 70, 75, 80, 90, 100, 125, 150, 175, 200, 225, 250, 275, 300, 350, 400, 450, 500, 550, 600, 650, 700, 750, 800, 900, 1000, 1200, 1400, 1600, 1800, or 2000 Joules (J). In some embodiments, the FUS energy applied by the transducer is no more than 5000, 4000, 3000, 2500, 2250, 2000, 1900, 1800, 1700, 1600, 1500, 1400, 1300, 1250, 1200, 1150, 1100, 1050, 1000, 950, 900, 850, 800, 750, 700, 650, 600, 550, 500, 450, 400, 350, 300, or 250 J. In some embodiments, the FUS energy applied by the transducer is between 10 J and 2000 J. In some embodiments, the FUS energy applied by the transducer is between 20 J and 1500 J. In some embodiments, the FUS by the transducer energy applied is between 50 J and 1250 J. In some embodiments, the FUS energy applied by the transducer is between 100 J and 1250 J. In some embodiments, the FUS energy applied by the transducer is between 250 J and 1250 J. In some embodiments, the FUS energy applied by the transducer is between 500 J and 1250 J. In some embodiments, the FUS energy applied by the transducer is about 200 J.

The duration of sonication can vary depending on the subject, the particular type and stage of the malignant tissue, the location and amount of the malignant tissue, and the degree to which the malignant tissue takes up sonosensitizer (and, in the case of 5-ALA, accumulates protoporphyrin-IX). In some embodiments, the malignant tissue is sonicated at multiple points, for example, at multiple points within a tumor. As used herein, a “point” refers to an FUS focal point and the tissue surrounding the point that is affected by the FUS. By sonicating points distributed throughout the malignant tissue, one can achieve a more even and constant effect throughout the tumor volume. This also permits one to use a lower total energy, which reduces the possible rise in temperature (and with it, the possible risk to surrounding normal tissue). In some embodiments, malignant tissue is sonicated at individual points that together expose all of the malignant tissue to FUS. In some embodiments, the points overlap. The points can be sonicated simultaneously, individually, or in groups. For example, in a treatment that includes targeting 18 points, all 18 points can be sonicated simultaneously, or the points can be sonicated sequentially, or in a random order, or in groups such as, for example, in pairs or triplets, or groups of other sizes. Where groups are sonicated, the groups can be physically grouped, or distributed to non-adjacent regions. In some embodiments, the malignant tissue is sonicated at 1, 2, 3, 4, 5, 6, 7, 8, 9, 10, 11, 12, 15, 20, 25, or 30 individual points, or at any value from 1 to 30. In some embodiments, the malignant tissue is sonicated at no more than 30, 25, 20, 19, 18, 17, 16, 15, 14, 13, 12, 11, or 10 individual points.

In some embodiments, the total duration of the pulse sequence is at least about 10 seconds, at least about 20 seconds, at least about 50 seconds, at least about 100 seconds, at least about 200 seconds, at least about 500 seconds, at least about 900 seconds, or at least about 1,200 seconds. In some embodiments, the total duration of the pulse sequence is from about 10 seconds to about 1200 seconds, such as from about 20 seconds to about 100 seconds, from about 20 seconds to about 500 seconds, from about 10 seconds to about 100 seconds, or from about 20 seconds to about 1,200 seconds. In some embodiments, the total duration of the pulse sequence is about 0.0001 second, about 0.0002 second, about 0.0005 second, about 10 seconds, about 20 seconds, about 50 seconds, about 100 seconds, about 200 seconds, about 500 seconds, about 1,000 seconds, or about 1,200 seconds. For example, the total duration of the pulse sequence can be about 100 seconds.

In some embodiments, the duration of each of the sonication pulses is at least about 0.0001 second, at least about 0.0002 second, at least about 0.0005 second, at least about 0.001 second, at least about 0.002 second, at least about 0.005 second, at least about 0.01 second, at least about 0.02 second, at least about 0.05 second, at least about 0.1 second, at least about 0.2 second, at least about 0.5 second, or at least about 1 second. In some embodiments, the duration of the sonication pulse is from about 0.0001 second to about 1 second, such as from about 0.0002 second to about 0.2 second, from about 0.0002 second to about 0.02 second, from about 0.0001 second to about 0.1 second, or from about 0.0005 second to about 0.05 second. In some embodiments, the duration of the sonication pulse is about 0.0001 second, about 0.0002 second, about 0.0005 second, about 0.001 second, about 0.002 second, about 0.005 second, about 0.01 second, about 0.02 second, about 0.05 second, about 0.1 second, about 0.2 second, or about 0.5 second. For example, the duration of the sonication pulse can be about 0.002 second.

The sonication can be continuous, or “cyclic.” In cyclic sonication, periods of exposure to focused ultrasound (“sonication periods”) are interspersed with rest periods, with no sonication. In some embodiments, the sonication includes at least one rest period. The sonication duration and the rest period can be the same or different lengths of time. In some embodiments, the sonication duration is longer than the rest period. In some embodiments, the sonication duration is shorter than the rest period. In some embodiments, the rest period is at least about 0.1 second, at least about 0.2 second, at least about 0.5 second, at least about 1 second or at least about 10 seconds. In some embodiments, the rest period is from about 0.1 second to about 10 seconds, such as from about 0.2 second to about 2 seconds, or from about 0.5 second to about 5 seconds. In some embodiments, the rest period is about 0.1 second, about 0.2 second, about 0.5 second, about 1 second, about 2 seconds, or about 5 seconds. For example, the rest period can be about 1 second. In some embodiments, the sonication is cyclic, with a sonication duration of about 0.002 second and a rest period of about 1 second.

In a cyclic sonication protocol, the rest period can also be measured by a pulse repetition frequency. In some embodiments, the pulse repetition frequency is from about 0.1 Hz to about 50 Hz, such as from about 0.2 Hz to about 2 Hz, from about 0.1 Hz to about 3 Hz, from about 0.5 Hz to about 5 Hz, from about 1 Hz to about 10 Hz, from about 0.5 Hz to about 20 Hz, or from about 0.1 Hz to about 50 Hz. In some embodiments, the pulse repetition frequency is about 0.1 Hz, about 0.5 Hz, about 1 Hz, about 2 Hz, about 3 Hz, about 5 Hz, about 10 Hz, about 20 Hz, or about 50 Hz. For example, the pulse repetition frequency can be about 1 Hz.

In various embodiments, malignant tissue is selectively destroyed without significant clinical effect on non-malignant tissue present at the ultrasound focus. In some embodiments, less than about 25%, less than about 20%, less than about 15%, less than about 10%, less than about 5%, less than about 4%, less than about 3%, less than about 2%, less than about 1% of the non-malignant tissue present at the ultrasound focus is damaged. In some embodiments, about 1%, about 2%, about 3%, about 4%, about 5%, about 10%, about 15%, about 20%, or about 25% of the non-malignant tissue present at the ultrasound focus is damaged. The amount of tissue damage can be determined using methods known to those of ordinary skill in the art, for example using MRI. In some embodiments, the temperature of the malignant tissue is raised by no more than 15° C., no more than 14° C., no more than 13° C., no more than 12° C., no more than 11° C., no more than 10° C., no more than 9° C., no more than 8° C., no more than 7° C., no more than 6° C., no more than 5° C., no more than 4° C., no more than 3° C., no more than 2° C., or no more than 1° C.

The ultrasound can be focused on the malignant tissue or can be focused on a broader volume that includes the malignant tissue or can be focused on an even broader volume that shows no signs of malignant tissue. Treatment with a sonosensitizer renders the malignant tissue more susceptible to SDT, making it possible to destroy malignant tissue without undue damage to non-malignant tissue included in the focus volume. For example, the tumor and a volume around it can be sonicated. Additionally, one can sonicate a complete anatomic region of the brain, such as, for example without limitation, a temporal lobe, a parietal lobe, a frontal lobe, an occipital lobe, the thalamus, the pituitary gland, the pons, the corpus callosum, the basal ganglia, the brainstem, an entire hemisphere, the supratentorial region, the infratentorial region, and the like. Additionally, one can sonicate a part or the whole of the brain FLAIR region (fluid-attenuated inversion recovery—an MRI technique designed to remove the signal from liquids in the brain). The methods of the disclosure can also in conjunction with surgical resection of a tumor, for example to treat the resulting tumor cavity to eliminate any malignant cells not removed by the resection.

In some embodiments, the position of the tumor is located using MRI. In some embodiments, the tumor is located using X-ray imaging. In some embodiments, the tumor is sonicated. In some embodiments, the tumor and a volume around the tumor is sonicated. In some embodiments, the tumor and a margin extending from the tumor surface by 0.2, 0.5, 1, 2, 3, 4, 5, 6, 7, or 8 cm is sonicated. In some embodiments, a complete anatomic region of the brain undergoes sonication. In some embodiments, a temporal lobe, a parietal lobe, a frontal lobe, an occipital lobe, the thalamus, the pituitary gland, the pons, the corpus callosum, the basal ganglia, the brainstem, an entire hemisphere, the supratentorial region, or the infratentorial region is sonicated. In some embodiments, the brain FLAIR region is sonicated. In some embodiments, two or more anatomical regions are sonicated. In some embodiments, the tumor is resected, and the tumor cavity is sonicated to eliminate residual malignant tissue or cells. In some embodiments, the tumor cavity is sonicated to a depth of 0.2, 0.5, 1, 2, 3, 4, 5, 6, 7, or 8 cm.

In some embodiments, treatment, including the administration of the sonosensitizer and sonication of malignant tissue, is repeated at a treatment interval or at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 15 days, at least about 16 days, at least about 18 days, at least about 20 days, at least about 21 days, at least about 24 days, at least about 25 days, at least about 26 days, at least about 28 days, at least about 30 days, at least about 35 days, at least about 40 days, at least about 45 days, at least about 50 days, at least about 55 days, at least about 60 days, at least about 65 days, at least about 70 days, at least about 75 days, at least about 80 days, at least about 85 days, or at least about 90 days. In some embodiments, the treatment repetition interval is less than about 120 days, less than about 110 days, less than about 100 days, less than about 90 days, less than about 80 days, less than about 70 days, less than about 60 days, less than about 50 days, less than about 40 days, less than about 30 days, less than about 20 days, less than about 14 days, less than about 10 days, less than about 7 days, less than about 6 days, less than about 5 days, less than about 4 days, less than about 3 days, or less than about 2 days.

The subject of treatment may be a mammal, e.g., a human or a non-human mammal, for example a companion animal, such as a dog, cat, rat, or the like, or a farm animal, such as a horse, donkey, mule, goat, sheep, pig, or cow, and the like. In some embodiments, the subject is human.

Treatment may involve selectively inducing apoptosis, autophagy, necrosis and/or senescence within malignant tissue in a subject, e.g., by providing an effective amount of sonosensitizer to the malignant tissue, and sonicating the tissue using a focused ultrasound device at a pulse duration of from about 0.1 millisec to about 10 millisec, at a pulse repetition frequency of from about 0.1 Hz to about 50 Hz, at an intensity at the ultrasound beam focus of from about 5 W/cm² to about 10,000 W/cm², an a total duration of the pulse sequence of 10 to 1,200 seconds using the methods and parameters set forth above. In certain embodiments, a pulse duration of about 2 millisec, at a pulse repetition frequency of about 1 Hz, and an intensity at the ultrasound beam focus of about 100 W/cm², and a total duration of the pulse sequence of 100 seconds can be used.

It can occur that sonication is performed by a person other than a treating physician. In order to minimize risks, and insure that treatment is performed appropriately, a container may be used to key operation of the FUS device to the subject to be treated. In some embodiments, the sonosensitizer formulation is provided in a container that comprises a machine-readable identifier, wherein the identifier identifies the contents of the container, the source of the formulation, the amount of the formulation, the subject to which the formulation is to be administered, the focused ultrasound treatment prescribed for the subject (for example, specifying the ultrasound frequency, power, energy, duration, or a combination thereof), an identification code or serial number, or a combination thereof. The machine-readable identifier can be encrypted, in order to preserve confidential patient information. In some embodiments, the container is sufficient to contain an effective amount of sonosensitizer, an effective amount of a potentiating agent, and/or an effective amount of microbubbles. In some embodiments, the machine-readable identifier is a bar code, QR code, or RFID device. In some embodiments, the focused ultrasound device includes a device for reading the machine-readable identifier. In some embodiments, the machine-readable identifier is encrypted. In some embodiments, the FUS device is locked in the absence of an appropriate machine-readable identifier. In some embodiments, the FUS device treatment parameters are programed via the machine-readable identifier.

### EXAMPLES

The following preparations and examples are given to enable those skilled in the art to more clearly understand and to practice the present invention. They should not be considered as limiting the scope of the invention, but merely as being illustrative and representative thereof.

### Example 1

### Study Drug

Six hours prior to MRgFUS, adult patients with recurrent GBM are administered SONALA-001 (10 mg/kg), an investigational IV formulation of 5-ALA. Oral 5-ALA is an FDA- and EMU-approved agent used in fluorescence-guided surgery to visualize glioma infiltration and maximize extent of resection.

### MR-Guided Focused Ultrasound

The INSIGHTEC EXABLATE Model 4000 Type-2 system is an investigational MRgFUS with a 1000-element transducer utilizing real-time MR imaging during treatment delivery. It includes the same monitoring capabilities as the ablative, FDA-approved Type-1 system for essential tremor and tremor-dominant Parkinson's Disease. However, the Type-2 device can target a large range of brain anatomy and operates at relatively low ultrasound frequencies and intensities, preventing tissue heating when inducing sonoluminescence. Intra-procedural MR thermometry automatically triggers an auto-stop if any tissue approaches ablative temperatures. At the 200 J output energy level described below, the MRgFUS power is 50 W (measured at the transducer surface) at 90 seconds with 4% duty cycle, with a pulse duration of 0.002 second, a pulse repetition frequency of 1 Hz, and target intensity of 100-150 W/cm² depending on the attenuation of the particular patient's skull.

### Study Design

This open-label, single-center, first-in-human Phase 0/1 study examines escalating energy doses of 5-ALA SDT in up to 30 adult patients with recurrent GBM undergoing a planned re-resection. Patients must have completed standard-of-care chemotherapy and radiotherapy prior to radiographic recurrence. In a Dose-Escalation Arm, 9-18 patients are assigned to one of three ascending acoustic energy doses of MRgFUS (200 J/400 J/800 J, measured at transducer surface), followed by a four-day interval until tumor resection. In each patient, half the tumor volume, including Gadolinium-enhancing and non-enhancing tumor, is targeted with MRgFUS and the other half serves as an internal control. Using the tumor pharmacodynamic endpoints described below, the Minimum Biological Dose (MBD) associated with 5-ALA SDT response is identified. In a subsequent Time-Escalation Arm, 12 patients are treated at the MBD and assigned to one of two time-intervals between SDT and resection.

### Study Patient Imaging

Preoperative physiological MR imaging is conducted to minimize the risk of sampling bias, including avoidance of tissue regions that are nonviable and/or represent radiation necrosis. Twenty-four hours pre- and post-5-ALA-SDT, all patients undergo MRI at 3T (Ingenia, Philips Healthcare, Best, Netherlands) and, during MRgFUS administration, all patients are imaged at 1.5T (General Electric Healthcare Excite HDXt, Waukesha, WI). In addition to structural MR imaging (e.g., pre- and post-contrast 3D T1 weighted imaging, FLAIR T2), dynamic susceptibility contrast (DSC) MRI, and diffusion weighted imaging (DWI) are performed 24-hours pre- and post SDT. Acquisition and analysis protocols for DSC-MRI and DWI followed current best practices. Collectively, these image acquisitions not only enable tumor targeting, but also quantification of treatment-induced changes in relative cerebral blood volume (rCBV) using DSC-MRI and apparent diffusion coefficient (ADC) using DWI-MRI.

Image analysis was performed using IBRadTech (Imaging Biometrics, Milwaukee, WI) and in-house Matlab software (Mathworks). Pre- and post-treatment regions of interest (ROIs) were drawn to delineate the contrast agent enhancing and non-enhancing regions, the SDT-treated tumor volume, and the SDT-untreated tumor volume. All ROIs were drawn by a neuroradiologist specialized in neuro-oncologic imaging. Summary statistics of the parameter percent changes in each ROI was computed.

### Glioblastoma Pharmacodynamics

For each patient, en bloc resection of enhancing and nonenhancing tumor was completed, enabling serial histological reconstruction of treated and non-treated regions. A tumor pharmacodynamic response was defined as a two-fold increase in the percentage of apoptotic cells in treated enhancing tissue vs. adjacent non-treated control. Predetermined tissue samples from each patient were acquired using intraoperative neuronavigation and tested for oxidative stress biomarkers using validated assays for 4-hydroxynonenal (4-HNE), glutathione (GSH), glutathione disulfide (GSSG), cysteine (Cys), cystine (CySS), and total reduced thiols. Small molecular weight thiols (GSH and Cys) and disulfides (GSSG and CySS) were measured by liquid chromatography tandem mass spectrometry (LC-MS/MS). The levels of total reduced thiols were measured spectrophotometrically.

Fresh frozen paraffin-embedded (FFPE) tissues were stained with anti-4HNE (Adipogen, HNEJ-2, 1:100), anti-MIB-1 (DAKO, M724029, 1:100), anti-activated caspase-3 (Cell Signaling, #9661, 1:1000) and anti-pH2AX (Cell Signaling, #9718, 1:2000) using standard immunohistochemistry protocols with the BOND RX automated system (Leica Biosystems, Wetzlar, Germany). The stained slides were imaged using the Leica Versa microscope and analyzed using APERIO image-analysis software. Percentage of positive cells were quantified from random selection of at least 12 regions of interest for each tissue section from both FUS-treated and control regions. All slides and images were analyzed by a board-certified neuropathologist.

### Study Drug Pharmacokinetics

LCMS/MS methods were developed to determine the levels of 5-ALA and PPIX in plasma and blood following IV administration of SONALA-001. Samples were analyzed using SCIEX ExionLC UHPLC system and the detection was performed on SCIEX QTRAP 6500+ mass spectrometer by multiple reaction monitoring mode to monitor the precursor-to-product ion transitions in positive electrospray ionization mode.

### Statistical Analysis

To characterize patient demographic and clinical characteristics, as well as tumor pharmacokinetic and pharmacodynamic measurements, descriptive statistics were performed with means/standard deviation and median, range for continuous data and frequencies and proportions for categorical data. Box plots were provided to compare PD biomarkers between SDT-treated and untreated control. R software (Version 4.0.5) were used to produce the boxplots and descriptive statistics.

### Results

### Study Patient Demographics

Three adult patients (ages, 44-68) accrued to the first energy dose-level (200J) of 5-ALA SDT. All presented with recurrent glioblastoma following standard-of-care temozolomide plus fractionated radiotherapy and consented to re-resection as part of routine management of their tumor recurrence. The median Gadolinium-enhancing tumor volume was 22.5 cm³ (range, 2.2-65.7 cm³) and the median ECOG Performance Status was 1 (range, 0-3). The median clinical follow-up was 4 weeks.

### Drug and Device Safety

All patients underwent 5-ALA SDT without serious drug- or device-related adverse events. One patient experience transient, subclinical hypotension within one hour of Sonala-001 infusion. In this case, systolic blood pressure lowered from the 130s to the 90s, but self-corrected with increased PO fluid intake. Resection sites displayed hypersensitivity to pain during frame placement requiring stronger pain management. One patient experienced nausea in the MRI and subsequent emesis required early stopping of the procedure. No patient demonstrated 5-ALA-related photosensitivity. MRgFUS was administered to all patients without device-related adverse events. Based on structural and physiological MR imaging, no radiographic changes related to non-targeted brain tissue were detected.

### Imaging Characteristics

Physiologic parameter changes were spatially heterogenous (e.g., regional increases in ADC) and patient-dependent. Across the three patients, the range of median percent changes in ADC for the treated and control regions of interest was-0.4 to 7.0% and −0.1 to 9.2%, respectively. The range of median percent changes in the DSC-MRI parameter, CBV, was −27.2 to 31.8% and −16.1 to 7.9%, respectively.

The first patient treated with 5-ALA SDT was a recurrent glioblastoma patient who presented with tumor recurrence within a right frontal lobe resection cavity. The posterior half of this gadolinium-enhancing tumor was targeted with 5-ALA SDT and the anterior half was designated as an untreated control. Six hours following administration of SONALA-001, planning software for the INSIGHTEC EXABLATE Model 4000 Type-2 system was combined with real-time MR imaging to deliver 200 J of non-ablative energy to the target volume. No energy was delivered to the non-targeted control region of the tumor. Intra-procedural MR thermometry confirmed non-ablative tissue temperatures during the treatment.

Pre- and post-treatment MRI apparent diffusion coefficient (ADC) maps were generated 24 hours before and after 5-ALA SDT. Observed increases in ADC signal were more prominent in the treated regions of the tumor, suggesting cellular fluid shifts induced by the therapy.

No microhemorrhages were observed in all post-treatment MRI susceptibility-weighted imaging (SWI).

### Pharmacokinetics of 5-ALA and PPIX in Plasma and Blood

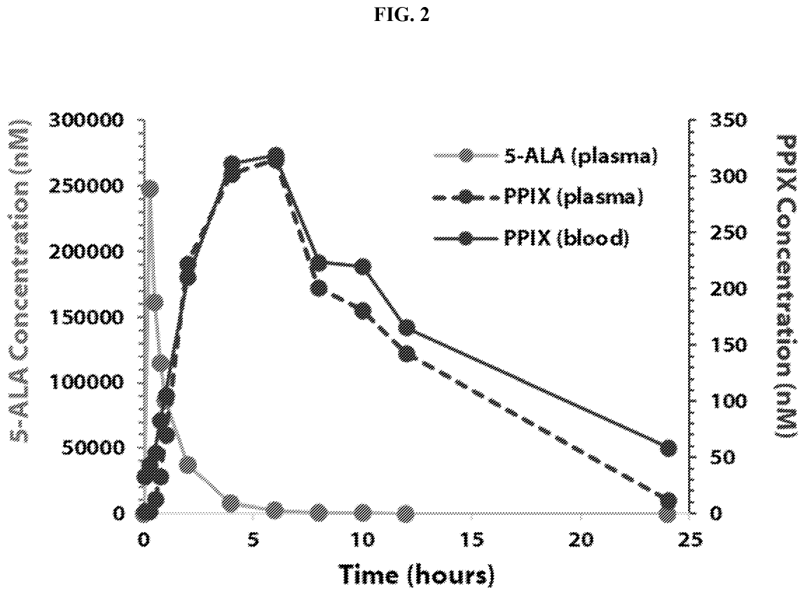

The median Cmax for 5-ALA in plasma was 307 μmol/L measured at 15 min post 10 mg/kg IV Sonala-001 administration (FIG. 2). PPIX levels began to elevate in both plasma and blood within 15 min of 5-ALA dosing and peaked at approximately 6 hours. The median Cmax of PPIX in plasma and blood was observed at 165 nmol/L and 319 nmol/L, respectively. Both 5-ALA and PPIX returned to endogenous levels 24 hours post Sonala-001 dosing.

### Tumor Cytotoxicity and Cytoarchitectural Effects

In all patients, the apoptosis biomarker cleaved caspase-3 was significantly increased in treated tumor vs. control (median, 48.6% vs. 29.6%, p=0.05). No significant differences in the percentage of MIB-1 (+) cells or cells with DNA damage (pH2AX) were observed in treated vs. control tissues. No cytoarchitectural changes to non-targeted tissue were detected in each patient's serial histological reconstruction.

5-ALA sonodynamic therapy (SDT) combines IV administration of 5-ALA (SONALA-001) with MRgFUS. Without being bound to theory, aberrant tumor metabolism of 5-ALA is believed to lead to protoporphyrin-IX (PPIX) accumulation and this molecule is activated by non-ablative ultrasound energy (200 J) to precipitate reactive oxygen species (ROS) formation. Four primary metrics of ROS formation are 4-hydroxynonenal (4-HNE), the glutathione (GSH) to glutathione disulfide (GSSG) ratio, and the cysteine (Cys) to cystine (Cyss) ratio, and total reduced thiols. ROS interactions with lipids result in lipid peroxidation, which plays a role in protoporphyrin phototoxicity. While lipid peroxidation has been shown to cause similar damage to DNA as radiation with cluster damage and double strand breaks, it has also been shown to damage phospholipids directly and furthermore act as cell death signal promoting different types of cell death such as apoptosis, autophagy and ferroptosis. Oxidized phospholipids are furthermore directly related to inflammatory diseases, frequently mediating a proinflammatory change. These events are understood to peak within days of 5-ALA SDT, while treatment-induced tumor cell death (measured by cleaved-caspase-3 [CC3]) can begin several days later. In this study, tissue acquisition occurred at Day 4 post-SDT.

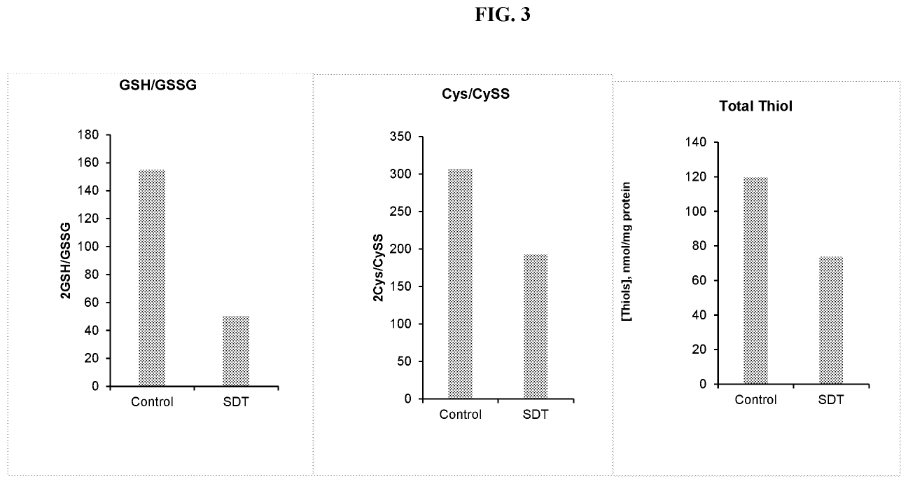

Serial histological reconstruction for Patient 1 demonstrated significantly higher levels of 4-HNE and CC3 within the treated region of the tumor as compared to adjacent non-treated control. Some evidence of heightened tumor vacuolization was also evident in treated regions on hematoxylin and eosin (H&E) staining. In comparison to non-treated control, 5-ALA SDT-treated tumor tissue in Patient 1 demonstrated significantly decreased ratios of 2GSH/GSSG and 2Cys/CySS, as well as a reduction in total thiol concentration, indicating targeted reactive oxygen species formation (FIG. 3).

### Reactive Oxygen Species Formation

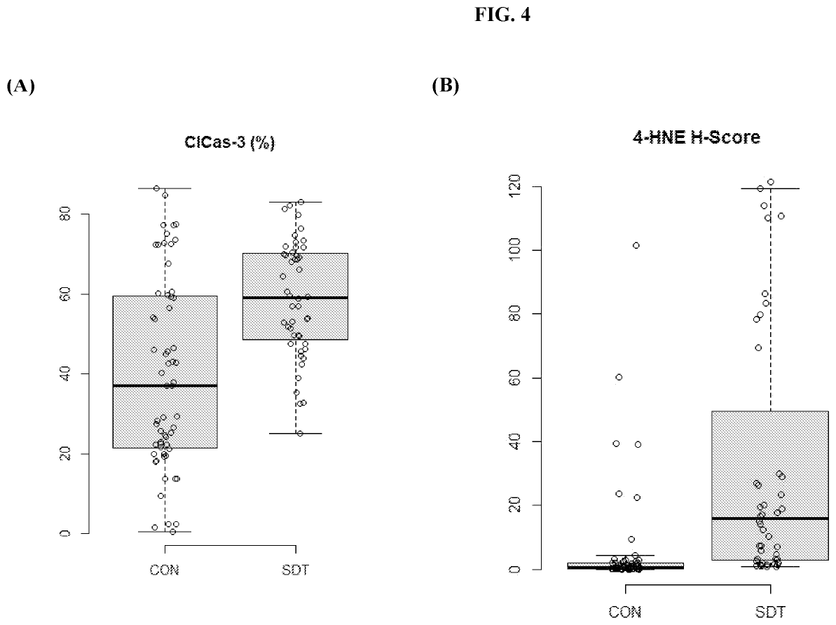

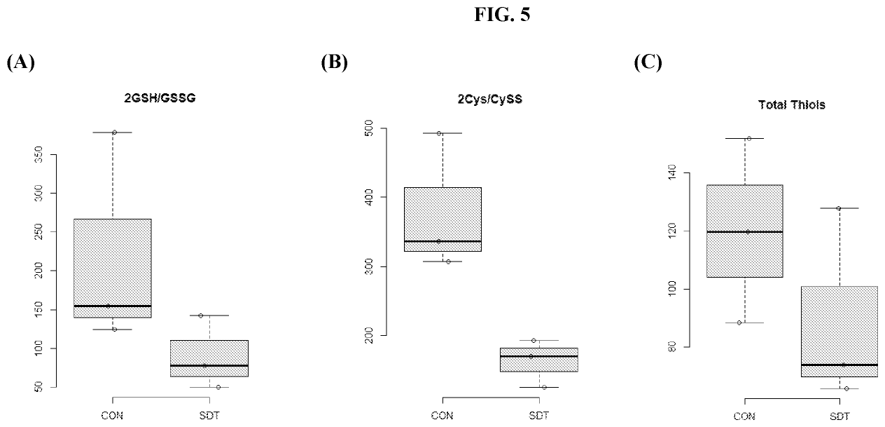

The oxidative stress biomarker for lipid peroxidation, 4-HNE, was significantly increased in treated tumor vs. control (FIG. 4). Levels of reduced thiols (GSH, Cys, total) were significantly decreased, while levels of their oxidized counterparts (GSSG and CySS) were significantly elevated in treated tumor vs. control. Such shifts in thiol content resulted in significant reduction of GSH/GSSG and Cys/CySS ratios in treated tissue, suggesting engagement in an oxidative event (FIG. 5).

### Example 2

Performed similarly to Example 1 with same study drug but for Diffuse Intrinsic Pontine Glioma (DIPG) and with typically only radiation therapy performed prior.

### MR-Guided Focused Ultrasound

Performed similarly to Example 1 but with an increase in the number of subspots per sonication (32 subspots). Targets were overlapped with a 2.0-3.0 mm spot to spot separation and Duty Cycle within 4-32% throughout the study. Pulse duration ranged from 0.0002 to 0.002 second.

### Study Design

Similar to Example 1, this multi-center, non-randomized, open label, first-in-pediatric Phase 1/2 study is a drug dose (5, 10, or 15 mg/kg SONALA-001) and focused ultrasound energy (200J, 400J, or 800J) escalation study containing 18-24 subjects, 9 cohorts of approximately 3 patients each. These pediatric patients with DIPG must have completed standard-of-care radiotherapy prior to radiographic treatment. No resection due to location but biopsy can be obtained. In the first patient, half of the Pons volume is targeted with MRgFUS and the other half serves as an internal control for safety. If no DLTs or any grade intracranial hemorrhage and any clinical symptoms (CTCAE, Version 5.0, i.e., intracranial hemorrhage Grades 2-5) are observed, the next patients will receive treatment over the entire pons. The intervention model will be sequential assignment using a Bayesian optimal interval (BOIN) dose escalation method. This method will be used to determine the Maximum Tolerated Dose, MTD-C, of the drug SONALA-001 and the EXABLATE Type-2 Investigational device combination. As such the primary outcome measures are the safety of ALA and SDT, followed by the frequency of clinically significant adverse events (DLT). Blood draws and Imaging will also be performed to obtain RAPNO and the pharmacokinetics of the drug.

### Study Patient Imaging

Preoperative physiological MR imaging is conducted to minimize the risk of sampling bias, including avoidance of tissue regions that are nonviable and/or represent radiation necrosis. Scans are divided into those that are collected for ORR (RAPNO) and those collected for the specific purposes of ALA SDT. Twenty-four hours pre- and post-5-ALA-SDT, all patients undergo MRI with the system and parameters as consistent as possible for the site. In addition to structural MR imaging (e.g., pre- and post-contrast 3D T1 weighted imaging, FLAIR T2), SWAN/SWI, dynamic susceptibility contrast (DSC) MRI, and diffusion weighted imaging (DWI) are performed 24-hours pre- and post SDT, along with 1 month post SDT. Acquisition and analysis protocols for DSC-MRI and DWI followed current best practices. Collectively, these image acquisitions not only enable tumor targeting, but also quantification of treatment-induced changes. MR perfusion with performed dynamic contrast enhanced (DCE) MRI and dynamic susceptibility contrast (DSC) MRI will be obtained to evaluate for changes in vessel density and vascularity that may reflect treatment response. Diffusion weighted imaging (DWI) and diffusion tensor imaging (DTI) will be obtained to evaluate for changes in cell density and extracellular water that may reflect tumor response. By collecting these datasets before (as part of routine surveillance scans) and after ALA SDT (during the post-treatment scan on Day 2/3 and Day 31/32 if applicable), the treatment induced changes in BBB permeability, perfusion and cellularity can be quantified and mapped regionally. T1 maps and susceptibility weighted imaging (SWI) will be acquired immediately before and after MRgFUS application to detect regional changes in oxidative stress and free radical formation. RAPNO measurements will be obtained on SOC MRI scans before SDT and at select subsequent follow-up scans. Follow-up scans can be done locally and confirmed by central review. The RAPNO assessment will document measurable disease with the 2D product of the largest perpendicular diameters (using T2-weighted or FLAIR sequences).

### Neurologic Examination

Neurologic examination for RAPNO evaluation includes assessments of mental status, motor-sensory, cerebellar/gait, cranial nerves. Brief neurological exams will note any new findings. Examinations are completed by appropriately licensed personnel as per institutional guidelines or as delegated by the Investigator. Abnormalities identified at the Screening Visit (Visit 1) will be documented in the subjects' source documents and on the medical history CRF. Changes will be captured as AEs on the AE CRF page, as deemed by the Investigator.

### Study Drug Pharmacokinetics

ALA and PpIX in plasma will be determined to derive the concentration of the ALA IV formulation by a validated HPLC-MS/MS method. All analyses will be performed by a designee of the Sponsor as described in Example 1.

### Statistical Analysis

The determination of MTD relies on the Bayesian Optimal INterval (BOIN) and its implementation is carried out using the software entitled “R Package for Designing Single-Agent and Drug-Combination Dose-Finding Trials Using Bayesian Optimal Interval Designs,” Zhou, 2021). The study is exploratory in nature and is not powered for inferential statistics, rather the main focus is estimation of key parameters related to the primary and secondary efficacy variables (including PK parameters). All available assessments of RAPNO response will be used to derive efficacy endpoints (response rate and duration of response). Confidence intervals will be 90% unless stated otherwise. All statistical analyses will be performed using R Software (R version 4.0.2 (2020-06-22)). All data recorded in eCRFs, as well as any outcomes derived from the data, will be presented in summary tables and/or data listings. Missing or invalid data will not be replaced. The confidence level for interval estimates will be 90%. For change from baseline calculations, “baseline” refers to the last measurement obtained prior to the first administration of study drug. Study Day 1 is the first day of study treatment (combination SONALA-001 and EXABLATE Type-2 device). Demographic data (including age, race, ethnicity, gender, height, weight, skull thickness, head circumference), medical history, prior treatments, and pre-treatment clinical characteristics will be summarized by treatment cohort. For categorical variables, frequencies and percentages will be presented. Continuous variables will be summarized using standard descriptive statistics (sample size, mean, median, standard deviation, minimum, maximum and range). Concomitant medication usage will be presented in individual subject listings. The number of subjects who enrolled into the study, the frequency and percentage of subjects who withdrew from the study, and the reasons for withdrawal, will be tabulated.

### Results

### Study Patient Demographics

Three pediatric patients (ages, 5-14) accrued to the first energy dose-level (200J) of 5-ALA SDT. All presented with DIPG following radiotherapy. The first and second patients were treated in a staged layout, a month between bilateral procedures, and the third patient was treated with a singular treatment to the entire pons. It is important to note that the second patient was compassionate use and treatment bilateral over the tumor including extensions beyond the pons.

### Drug and Device Safety

All patients underwent 5-ALA SDT without serious drug- or device-related adverse events. Due to anesthesia, procedural AE's due to hypothermia were possible but none reported and mitigation steps were performed using heated air around the patient in the MRI. This kept body temperatures between 33-35° C. for the procedures. No patient demonstrated 5-ALA-related photosensitivity. MRgFUS was administered to all patients without device-related adverse events. Based on structural and physiological MR imaging, no radiographic changes related to non-targeted brain tissue were detected nor noticeable damage to the treated areas that would indicate potential micro-hemorrhages.

### Tumor Progression

The first two patients had an objective reduction in the halves of pons or tumor treated. While no progression occurred in the first patient 1 month after the first side, there was some progression in the second patient 1 month post their first procedure. However, the progression was unilateral and only upon the regions not treated in the first procedure. No progression was observed 1 month after the whole pons treatment of the third patient. One month following the second side of patient 1 there was a significant reduction in tumor flair volume estimated to be between 35-50% overall. Compassionate use patient had some further progression in non-treated areas in the brain stem. One month follow-up for patient 3 is yet to be obtained.

### Clinical Observations

All three patients awoke from the procedure and reported increased energy for days to months after their procedure with improvements objectively observed by the parents. Two months post the first procedure of the first patient there is continued improvements in the patient's symptoms. One month post the third patient's procedure a clinically significant improvement was reported for the left cranial nerve 6 palsy. Compassionate patient, subject 2, reported continued improvements but still potential progression from testing.

The discussion of the general methods given herein is intended for illustrative purposes only. Other alternative methods and alternatives will be apparent to those of skill in the art upon review of this disclosure, and are to be included within the spirit and purview of this application. Throughout this specification, various patents, patent applications and other types of publications (e.g., journal articles, electronic database entries, etc.) are referenced. The disclosure of all patents, patent applications, and other publications cited herein are hereby incorporated by reference in their entirety to the same extent as if each individual publication or patent application was specifically and individually indicated to be incorporated by reference. No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art. The terms and expressions employed herein are used as terms and expressions of description and not of limitation, and there is no intention, in the use of such terms and expressions, of excluding any equivalents of the features shown and described or portions thereof.

What is claimed is:

1. A system for applying acoustic energy to a target region, the system comprising:
an ultrasound transducer comprising a plurality of transducer elements for providing a series of sonications to at least one target region; and
a controller configured to operate at least some of the transducer elements to apply focused ultrasound energy at the target region in the presence of a photodynamic agent, the ultrasound energy being applied in a sequence of pulses wherein each of the pulses has a duration of from about 0.1 millisec to about 100 millisec, and the pulses have a repetition frequency of from about 0.1 Hz to about 50 Hz, an intensity at the target region of from about 5 W/cm² to about 10,000 W/cm², a focal peak pressure of about 0.7 MPa to 20 MPa at the target region, and a total duration from 10 sec to 1,200 sec.

2. The system of claim 1, wherein the photodynamic agent is 5-aminolevulinic acid.

3. The system of claim 1, wherein the photodynamic agent is fluorescein.

4. The system of claim 1, wherein the total duration is from about 50 sec to about 500 sec.

5. The system of claim 1, wherein the intensity is from about 5 W/cm² to about 5000 W/cm².

6. The system of claim 1, wherein the intensity is from about 100 W/cm² to about 3000 W/cm².

7. The system of claim 1, wherein a total acoustic energy applied at the target region is between 10 J and 1000 J.

8. The system of claim 7, wherein the total acoustic energy applied at the target region is between 50 J and 500 J.

9. The system of claim 1, wherein the focal pressure is about 10 MPa.

10. The system of claim 1, wherein the controller is configured to cause the focused ultrasound energy to be applied to the target region at a plurality of individual points.

11. The system of claim 1, further comprising a magnetic resonance imaging device, operatively coupled to the controller, for locating the target region.

12. The system of claim 1, wherein the focal pressure is a maximum clinically tolerable pressure.

13. A method applying acoustic energy to a target region, the method comprising:
providing an effective amount of a photodynamic agent at the target region; and
applying focused ultrasound energy at the target region in the presence of the photodynamic agent, the ultrasound energy being applied in a sequence of pulses wherein each of the pulses has a duration of from about 0.1 millisec to about 100 millisec, and the pulses have a repetition frequency of from about 0.1 Hz to about 50 Hz, an intensity at the target region of from about 20 W/cm² to about 10,000 W/cm², a focal peak pressure of about 0.7 MPa to 20 MPa at the target region, and a total duration from 10 sec to 1,200 sec.

14. The method of claim 13, wherein the target region comprises or consists of malignant tissue.

15. The method of claim 14, wherein the malignant tissue is glioblastoma multiforme, optical pathway glioma, diffuse intrinsic pontine glioma, astrocytoma, ependymoma, medulloblasoma, oligodendroglioma, hemangioblastoma, rhabdoid tumor, brain metastases from another cancer (selected from breast adenocarcinoma, small cell lung carcinoma, non-small cell lung carcinoma, squamous cell lung carcinoma, metastatic malignant melanoma, and prostate carcinoma), meningioma, primary pituitary gland malignancy, malignant nerve sheath tumor, neurofibroma, cutaneous T-cell lymphoma (CTCL), noncutaneous peripheral T-cell lymphoma, lymphomas associated with human T-cell lymphotropic virus (HTLV), adult T-cell leukemia/lymphoma (ATLL), acute lymphocytic leukemia, acute nonlymphocytic leukemias, chronic lymphocytic leukemia, chronic myelogenous leukemia, Hodgkin's Disease, non-Hodgkin's lymphomas, multiple myeloma, neuroblastoma, retinoblastoma, Wilms' Tumor, bone tumors, soft-tissue sarcomas, infiltrating or metastatic squamous cell carcinoma, salivary gland tumors, nasopharyngeal carcinomas, oral, laryngeal, esophageal tumors, urethral cancer, ureteral cancer, renal cell cancer, bladder carcinoma, bladder carcinoma in situ, metastatic carcinoma of the prostate, bladder, renal, uterine, ovarian, testicular, cancers, uterine, cervical, and uterine carcinoma, rectal or colon cancer, lung cancer, mesothelioma, small cell lung carcinoma, non-small cell lung carcinoma, squamous cell lung carcinoma, breast cancer, gastric cancer, esophageal cancer, and colon carcinoma, cholangiocarcinoma, hepatic carcinoma, pancreatic adenocarcinoma, melanoma, infiltrating basal cell carcinomas, other skin cancers, liver cancer or thyroid cancer.

16. The method of claim 15, wherein the focused ultrasound energy induces a cytotoxic effect within the target region.
